(19) United States
(12) Patent Application Publication
(10) Pub. No.: US 2014/0170703 A1
(43) Pub. Date: Jun. 19, 2014

(54) RECOMBINANT MICROORGANISM

(71) Applicant: KAO CORPORATION, TOKYO (JP)

(72) Inventors: MASATOSHI TOHATA, TOCHIGI (JP); KAZUHISA SAWADA, TOCHIGI (JP); KATSUYA OZAKI, TOCHIGI (JP); KAZUO KOBAYASHI, TOCHIGI (JP); NAOTAKE OGASAWARA, TOCHIGI (JP)

(73) Assignee: KAO CORPORATION, TOKYO (JP)

(21) Appl. No.: 14/089,575

(22) Filed: Nov. 25, 2013

Related U.S. Application Data
(62) Division of application No. 10/578,613, filed on Mar. 12, 2007, now abandoned.

(30) Foreign Application Priority Data
Nov. 7, 2003 (JP) 2003-379167

(51) Int. Cl. C12N 15/75; C12P 21/00

(52) U.S. Cl. CPC C12N 15/75; C12P 21/00
USPC 435/69.1; 435/252.31

(57) ABSTRACT

A recombinant microorganism obtained by transferring, into a host microorganism capable of producing protein or polypeptide with increased productivity, a gene encoding a protein or polypeptide, and a method for producing a protein or polypeptide by use of the recombinant microorganism. The recombinant microorganism is prepared by transferring, to a mutant strain of microorganism from which any of Bacillus subtilis genes comA, yopO, treR, yvbA, cspB, yvaN, yttP, yurK, yozA, licR, sigL, mntR, glcT, yvdE, ykvE, slr, rocR, ccpA, yaaT, yyaA, yycH, yacP, hprK, rsiX, yhdK, and ylbO, or one or more genes functionally equivalent to any of these genes have been deleted or knocked out, a gene encoding a heterologous protein or polypeptide.

### TECHNICAL FIELD

The present invention relates to a recombinant microorganism which may be used to produce useful proteins or polypeptides, as well as to such proteins and polypeptides.

### TECHNICAL BACKGROUND

Microorganisms are widely used for industrially producing a broad range of useful substances, including alcoholic beverages, certain types of foods such as miso and shoyu, amino acids, organic acids, nucleic-acid-related substances, antibiotics, sugars, lipids, and proteins. These substances also find diversified uses, including foods, pharmaceuticals, detergents, products for daily use such as cosmetics, and a variety of chemical raw materials.

In industrial production of useful substances by use of microorganisms, improvement of productivity is one major topic of interest, and one approach therefor is breeding of microorganisms through mutagenesis or other genetic means. Recently, in particular, with advancement of microbial genetics and biotechnology, more efficient breeding of useful microorganisms is performed through gene recombination techniques, and in association therewith, host microorganisms for obtaining recombinant genes are under development. For example, Bacillus subtilis Marburg No. 168, which has already been confirmed to be safe and have excellent characteristics as a host microorganism, has been further improved.

However, microorganisms inherently possess diversified genes so that they can cope with environmental changes in the natural world, and thus, they do not necessarily exhibit high production efficiency of proteins or similar substances in industrial production, where only limited production media are employed.

### DISCLOSURE OF THE INVENTION

The present invention provides a recombinant microorganism prepared by transferring, to a mutant strain of microorganism from which any of Bacillus subtilis genes comA, yopO, treR, yvbA, cspB, yvaN, yttP, yurK, yozA, licR, sigL, mntR, glcT, yvdE, ykvE, slr, rocR, ccpA, yaaT, yyaA, yycH, yacP, hprK, rsiX, yhdK, and ylbO, or one or more genes functionally equivalent to any of these genes have been deleted or knocked out, a gene encoding a heterologous protein or polypeptide.

### BRIEF DESCRIPTION OF THE DRAWINGS

FIG. 1 schematically shows a method for preparing a DNA fragment for deleting a gene through SOE-PCR (SOE: splicing by overlap extension) (see Gene, 77, 61 (1989), and a method for deleting a target gene (replacing the target gene with a drug resistance gene) through use of the DNA.

### MODES FOR CARRYING OUT THE INVENTION

The present invention is directed to a recombinant microorganism obtained by transferring, into a host microorganism capable of producing protein or polypeptide with increased productivity, a gene encoding a protein or polypeptide, and to a method for producing a protein or polypeptide by use of the recombinant microorganism.

The present inventors have conducted extensive studies on, among many different genes encoded on the genome of a microorganism, genes which are not needed in or which are detrimental to the production of useful proteins or polypeptides, and have found that, when a gene encoding a target protein or polypeptide is transferred to a microorganism such as Bacillus subtilis after a specific gene is deleted or knocked out from the genome of the microorganism, productivity of the target protein or polypeptide is enhanced as compared with the case before the deletion or knocking out.

In the microorganism of the present invention, since genes which are unnecessary in or detrimental to the production or a target protein or polypeptide are deleted or knocked out, waste of culture media, including energy loss, production of byproducts, and reduced specific production rate, is significantly reduced, and in addition, protein and polypeptide can be produced over a prolonged period, whereby a target product can be produced with high efficiency.

In the present invention, homology between amino acid sequences and that between nucleic acid sequences are both determined by use of the Lipman-Pearson method (Science, 227, 1435 (1985)). Specifically, calculation is performed by use of a homology analysis program (Search Homology) developed by genetic information processing software, Genetyx-Win (Software Development Co., Ltd.), with ktup (the unit size to be compared) being set 2.

No particular limitation is imposed on a parent microorganism for constructing the microorganism of the present invention, so long as it has a gene which is not necessary for producing a target protein or polypeptide; specifically, any of the Bacillus subtilis genes or genes functionally equivalent thereto as shown in Table 1, wherein the gene may be of wild-type or a mutant. Specific examples include Bacillus subtilis and similar microorganisms belonging to the genus Bacillus, microorganisms belonging to the genus Clostridium, and yeast. Inter alia, microorganisms belonging to the genus Bacillus are preferred. In particular, Bacillus subtilis is preferred, from the viewpoint that complete genomic information of this microorganism has already been obtained, and thus genetic engineering techniques and genomic engineering techniques have been established, and that the microorganism has ability to secrete the produced protein extracellularly.

Examples of the target protein or polypeptide to be produced by use of the microorganism of the present invention include enzymes, physiologically active substances, and other proteins and polypeptides which find utility in foods, pharmaceuticals, cosmetics, detergents, fiber-treating agents, clinical assay agents, etc.

Taking Bacillus subtilis, which is known to have 4,106 genes on the genome, as an example, one or more genes which are to be deleted or knocked out are any of the Bacillus subtilis genes shown in Table 1, or are selected from among the genes functionally equivalent thereto. The present inventors have found that such genes do not directly participate in production of the target protein or polypeptide and are unnecessary for the growth of microorganism in ordinary industrial production media.

The names, numbers, and functions of respective genes in the Tables contained herein conform with the Bacillus subtilis genome data reported in Nature, 390, 249-256 (1997) and made public by JAFAN (Japan Functional Analysis Network for Bacillus subtilis; BSORF DB) on the Internet (http://bacillus.genome.ad.jp/, renewed Jun. 17, 2003).

TABLE 1

| Name of | | Functions or other information of the |
|---|---|---|
| the gene | Gene ID | gene |
| comA | BG10381 | two-component response regulator |
| yopO | BG13648 | deduced transcriptional regulator, |
| | | spβ prophage protein |
| treR | BG11011 | trehalose operon transcriptional |
| | | represser (GntR family) |
| yvbA | BG14078 | deduced transcriptional regulator (ArsR |
| | | family) |
| cspB | BG10824 | cold shock-related major factor |
| yvaN | BG14069 | deduced transcriptional regulator |
| yttP | BG13927 | deduced transcriptional regulator (TetR |
| | | family) |
| yurK | BG13997 | deduced transcriptional regulator (GntR |
| | | family) |
| yozA | BG13748 | deduced transcriptional regulator (ArsR |
| | | family) |
| licR | BG11346 | transcriptional regulator |
| | | (antiterminator), lichenan operon |
| | | (licBCAH) regulation |
| sigL | BG10748 | RNA polymerase σ factor (o54) |
| mntR | BG11702 | manganese transport regulator |
| glcT | BG12593 | transcriptional regulator essential to |
| | | expression of ptsGHI operon (BglG |
| | | family, antiterminator) |
| yvdE | BG12414 | deduced transcriptional regulator (LacI |
| | | family) |
| ykvE | BG13310 | deduced transcriptional regulator (MarR |
| | | family) |
| slr | BG11858 | transcriptional activator for |
| | | competence- or sporulation-related genes |
| rocR | BG10723 | transcriptional activator for arginine- |
| | | assimilating operon (NtrC family) |
| ccpA | BG10376 | carbon source catabolism reppression- |
| | | related transcriptional regulator (Lacl |
| | | family) |
| yaaT | BG10096 | type-II signal peptidase-like protein |
| yyaA | BG10057 | DNA-binding protein SpoOJ-like protein |
| yycH | BG11462 | Function unknown (homologous gene has |
| | | been found in other organisms) |
| yacP | BG10158 | Function unknown (homologous gene has |
| | | been found in other organisms) |
| hprK | BG14125 | Hpr protein Ser residue |
| | | phosphoenzyme/dephosphoenzyme |
| rsiX | BG10537 | anti σX factor |
| yhdK | BG13017 | Function unknown, related to repression |
| | | of σM factor expression |
| ylbO | BG13367 | expression regulator for gene in σE- |
| | | related metrocytein |

Genes derived from other microorganisms, preferably from bacteria belonging to the genus Bacillus, which have the same functions as any of the Bacillus subtilis genes shown in Table 1, or have 70% or more homology with the nucleotide sequence of any of the genes shown in Table 1, preferably 80% or more homology, more preferably 90% or more, further preferably 95% or more, yet more preferably 98% or more, should be interpreted to be functionally equivalent to the genes shown in Table 1, and thus to constitute the genes which are to be deleted or knocked out according to the present invention. In this connection, homology of nucleotides is computed by use of the Lipman-Pearson method (Science, 227, 1435, 1985).

Many of the genes shown in Table 1 which encode Bacillus subtilis are regulatory genes participating in activation or suppression of expression of a variety of genes, or genes deduced to be such regulatory genes. The present invention has been attained on the basis of this finding; i.e., the presence of regulatory genes unnecessary in or detrimental to production of protein or polypeptide has now been unveiled in the present invention.

Notably, attention is drawn to the fact that many of the listed “unnecessary” or “detrimental” genes are regulatory genes participating in sugar intake or metabolism, as exemplified by the glcT gene, which acts as an anti-terminator for a glucose PTS intake operon; the licT gene, which acts as an anti-terminator for a lichenan hydrolysis operon; the treR gene, which acts as a repressor of trehalose intake and metabolism; and the hprK gene and ccpA gene, which relate to glucose catabolite repression.

Also, in addition to the regulatory genes involved in sugar intake and metabolism, the rocR gene participating in activation of arginine assimilation, and competence-related comA gene and slr gene, which are also regulatory genes, may be deleted or knocked out, to thereby improve productivity of protein or polypeptide.

The genes shown in Table 1 include the yhdK gene, and the rsiX gene encoding the anti-ECF sigma factor which suppresses expression of an ECF sigma factor, sigma x. The yhdK gene has been reported to participate in suppression of sigma M (Mol. Microbiol., 32, 41, 1999). The sigL gene, which encodes sigma L, is also included in the genes of Table 1. This suggests that expression of a gene under regulation by sigma X or sigma M is favorable for production of protein, and conversely, some gene expression under regulation by sigma L is unfavorable.

By deleting or knocking out one or more genes selected from the above-mentioned genes, expression which is unnecessary in or harmful to the production of protein or polypeptide can be prevented, leading to enhanced productivity in such production of protein or polypeptide.

The number of gene(s) to be deleted or knocked out is one or more, preferably two or more, more preferably three or more, even more preferably 5 or more. When a microorganism of the present invention is constructed, deletion or inactivation of a gene or genes other than those mentioned above is possible. In such a case, a more improved effect is expected. An alternative method for achieving the present invention is inactivation, or knocking out, of a target gene by inserting thereto a DNA fragment of another origin or introducing a mutation to the transcription/translation-initiation region of the gene. Preferably, however, the target genes are physically deleted.

In an example procedure for deleting or knocking out the genes, any of the target genes shown in Table 1 is deleted or knocked out according to a plan which has been set up in advance. Alternatively, randomized deletion of genes or mutation by way of knocking out is performed, followed by evaluation on protein productivity and gene analysis.

The target gene may be deleted or knocked out through homologous recombination. That is, a DNA fragment containing a portion of the target gene is cloned with an appropriate plasmid vector to thereby obtain a ring-shaped recombinant plasmid, and the resultant plasmid is transferred into cells of a parent microorganism. Thereafter, through homologous recombination effected in a partial region of the target gene, the target gene on the genome of the parent microorganism is cleaved, thereby completing inactivation of the target gene. Alternatively, the target gene is knocked out by substitution or insertion of a base, or a linear DNA fragment containing a region outside the target gene sequence but not containing the target gene may be constituted through PCR or a similar method, and the thus-engineered gene or fragment is transferred into a cell of a parent microorganism. At two sites outside the mutation within the target gene in the genome of the parent microorganism genome, or at two regions outside the target gene sequence, double crossing-over homologous recombination is caused to occur, to thereby attain substitution with a gene fragment in which the target gene on the genome is deleted or knocked out.

Particularly when the parent microorganism used to construct the microorganism of the present invention is Bacillus subtilis, since several reports have already described methods for deleting or knocking out the target gene (see, for example, Mol. Gen. Genet., 223, 268 1990), repetition of any of such methods may be followed, to thereby produce a host microorganism of the present invention.

Randomized gene deletion or inactivation may be performed through use of a method similar to the above-described method for inducing homologous recombination by use of a randomly cloned DNA fragment, or by way of irradiation of a parent microorganism with gamma rays or similar rays.

Next will be described in more detail a deletion method employing double crossing over by use of a DNA fragment designed for the deletion purpose, the DNA fragment being prepared through SOE-PCR (Gene, 77, 61, 1989). However, in the present invention, the method for deleting genes is not limited to only the below-described method.

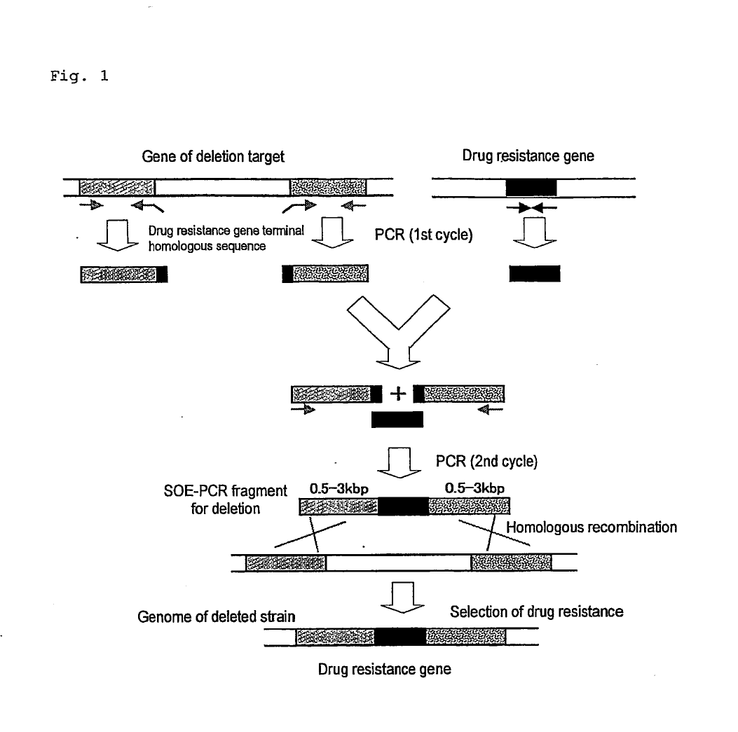

The DNA fragment use for the deletion purpose is a fragment constructed such that a drug resistant marker gene is inserted between a ca. 0.5 to 3 kb upstream sequence which flanks and is upstream of the gene to be deleted, and a ca. 0.5 to 3 kb downstream sequence which flanks and is downstream of the same gene. In the first cycle of PCR, the following three fragments are prepared: the upstream and the downstream fragments, which are to be deleted, and the drug resistant marker gene. The primers to be used in this step may, for example, be those specifically designed so that an upstream 10-30 base pair sequence of a drug resistance gene is added to the lower end of the upstream fragment, and a downstream 10-30 base pair sequence of the drug resistance marker gene is added to the upper end of the downstream fragment (FIG. 1).

Next, using three PCR fragments prepared in the first cycle as templates, the second cycle of PCR is performed by use of an upper primer of the upstream fragment and a lower primer of the downstream fragment. This step causes annealing with the drug resistance marker gene fragment in the sequence of the above-engineered drug resistance marker gene, and through PCR amplification, there can be obtained a DNA fragment with the drug resistance marker gene inserted between the upstream fragment and the downstream fragment (FIG. 1).

When a chloramphenicol-resistant gene is employed as a drug resistance marker gene, a DNA fragment for deleting a gene can be obtained through SOE-PCR under typical conditions described in literature (see, for example, PCR Protocols. Current Methods and Applications, Edited by B. A. White, Humana Press, pp. 251 (1993), Gene, 77, 61, 1989), by use of a primer set such as that shown in Table 2 and a conventional enzyme kit for PCR (e.g., Pyrobest DNA Polymerase (product of Takara Shuzo)).

When the thus-obtained DNA fragment for effecting gene deletion is introduced into cells through the competent method or a similar method, intracellular genetic recombination occurs in homologous regions which are present upstream and downstream of the gene to be deleted. Thus, cells in which the target gene has been substituted by a drug resistance gene can be selectively separated through employment of a drug resistance marker (FIG. 1). Specifically, when a DNA fragment for gene deletion prepared by use of a primer set shown in Table 2 is introduced into cells, colonies which have grown on an agar culture medium containing chloramphenicol are separated, and deletion of the target gene by way of substitution by the chloramphenicol-resistant gene is confirmed through an appropriate method such as PCR employing a genome as a template.

Subsequently, when a gene encoding a target protein or polypeptide is transferred to a host mutant microorganism strain from which any of the Bacillus subtilis genes shown in Table 1, or one or more genes selected from among the genes corresponding thereto has been deleted or knocked out, the microorganism of the present invention can be obtained.

No particular limitation is imposed on the gene encoding the target protein or polypeptide. Examples of the protein and polypeptide include physiologically-active peptides and enzymes for industrial purposes such as detergents, foods, fibers, feeds, chemicals, medicine, and diagnostic agents. Industrial enzymes may be functionally grouped into oxidoreductases, transferases, hydrolases, lyases, isomerases, and ligases/synthetases. Preferably, hydrolases such as cellulase, α-amylase, and protease may be used. Specific examples include cellulase belonging to family 5 in the classification of hydrolase (Bioche M. J., 280, 309, 1991); in particular, cellulase derived from a microorganism, more particularly cellulase derived from the genus Bacillus. Other specific examples of the types of industrial enzymes include alkaline cellulase which is derived from the genus Bacillus and has an amino-acid of SEQ ID NOs: 2 or 4, and cellulase which has an another amino-acid sequence having 70% homology with said amino-acid sequence, preferably 80% homology, more preferably 90%, further preferably 95%, still further preferably 98% or more.

Specific examples of α-amylase include α-amylase derived from a microorganism, preferably liquefied amylase derived from the genus Bacillus. More specific examples include alkaline amylase which is derived from the genus Bacillus and has an amino-acid sequence of SEQ ID NO: 6, and amylase which has another amino-acid sequence having 70% homology with said amino-acid sequence, preferably 80% homology, more preferably 90%, further preferably 95%, particularly preferably 98% or more. The homology of the amino-acid sequence is calculated by the Lipman-Pearson method (Science, 227, 1435 (1985)). Specific examples of protease include serine protease and metallo-protease which are derived from microorganisms, particularly those belonging to the genus Bacillus.

Preferably, a gene coding for a target protein or polypeptide has, on its upstream region thereof, one or more regulatory regions relating to transcription, translation, or secretion of the gene (specially, one or more regions selected from among a transcription initiation regulatory region including a promoter and a transcription initiation site; a translation initiation region including a ribosome-binding site and a start codon; and a secretion signal peptide region) properly ligated thereto. Preferably, it is preferred that three regions consisting of the transcription initiation regulatory region, the translation initiation regulatory region, and the secretion signal region be ligated to the target gene. Further preferably, the secretion signal peptide region is one that originates from the cellulase gene of a microorganism belonging to the genus Bacillus, and the transcription initiation region and the translation initiation region is a 0.6 to 1 kb region upstream of the cellulase gene. In one preferred example, a transcription initiation regulatory region, a translation initiation region, and a secretion signal peptide region of a cellulase gene derived from a microorganism belonging to the genus Bacillus disclosed in, for example, Japanese Patent Application Laid-Open (kokai) Nos. 2000-210081 and 190793/1990; i.e., a cellulase gene derived from KSM-S237 strain (FERM BP-7875) or KSM-64 strain (FERM BP-2886), is properly ligated to a structural gene of the target protein or polypeptide. More specifically, preferred DNA fragments to be ligated include a nucleotide sequence of base numbers 1 to 659 of SEQ ID NO: 1; a nucleotide sequence of base numbers 1 to 696 of a cellulase gene of SEQ ID NO: 3; a DNA fragment having a nucleotide sequence having 70% homology with any one of said nucleotide sequences, preferably 80% homology, more preferably 90%, further preferably 95%, even more preferably 98% or more; or a DNA fragment having a nucleotide sequence lacking a portion of any one of said nucleotide sequences. Preferably, one of these DNA fragments is properly ligated to a structural gene of the target protein or polypeptide. As used herein, a DNA fragment having a nucleotide sequence lacking a portion of any one of the above-mentioned nucleotide sequences is intended to mean a DNA fragment which has functions relating to transcription, translation, and secretion of the gene, without having a portion of any one of the above-mentioned nucleotide sequences.

The recombinant microorganism of the present invention can be obtained by a conventional transformation technique in which a recombinant plasmid containing a DNA fragment which includes a gene encoding the target protein or polypeptide, and is ligated to a proper plasmid vector is transferred into a host microorganism cell. Alternatively, the recombinant microorganism may be obtained making use of a DNA fragment prepared by ligating the above DNA fragment to a proper region which is homologous with a certain portion of the host microorganism genome, and inserted directly into a host microorganism genome.

The target protein or polypeptide obtained by use of the recombinant microorganism of the present invention may be produced in such a manner that a corresponding cell strain is inoculated onto a culture medium containing assimilable carbon sources and nitrogen sources, and other essential components; the cell strain is cultured through a conventional microorganism culturing method; and subsequently, protein or polypeptide is collected and purified.

Through the aforementioned procedure, a host mutant microorganism strain in which any of the Bacillus subtilis genes shown in Table 1 or one or more genes selected from genes functionally equivalent thereto have been deleted or knocked out can be engineered. In addition, by use of such a mutant strain, a recombinant microorganism can be produced. Thus, a useful protein or polypeptide can be effectively produced through employment of the mutant strain or the recombinant microorganism.

The method for constructing a recombinant microorganism according to the present invention, and the method for producing cellurase and α-amylase by use of the recombinant microorganism will next be described in detail, centering on working examples for constructing recombinant strain belonging to Bacillus subtilis from which the ccpA gene (BG10376) of Bacillus subtilis has been deleted.

### EXAMPLES

### Example 1

A genome DNA sample, serving as a template, extracted from Bacillus subtilis 168 strain and two primer sets (ccpA-AF and ccpA-A/CmR; and ccpA-B/CmF and ccpA-BR) shown in Table 2 were used to prepare a 0.6 kb fragment (A) flanking the upstream side of the ccpA gene on the genome and a 0.6 kb fragment (B) flanking the downstream side of the ccpA gene. A chloramphenicol-resistant gene of plasmid pC194 (J. Bacteriol. 150 (2), 815 (1982))) was inserted into the XbaI-BamHI cleavages site of plasmid pUC18, to thereby prepare a recombinant plasmid pCBB 31. The recombinant plasmid pCBB and a primer set consisting of CmF and CmR shown in Table 2 were used to prepare a 1 kb fragment (C) containing the chloramphenicol-resistant gene. Subsequently, SOE-PCR was performed by use of the primers ccpA-AF and ccpA-BR shown in Table 2, and by use of the thus-prepared three fragments (A), (B), and (C) in combination as templates, a 2.2 kb DNA fragment in which the fragments (A), (B), and (C) were ligated in this sequence was prepared (see FIG. 1). By use of the thus-prepared DNA fragment, Bacillus subtilis 168 strain was transformed through the competent method. Colonies grown in an LB agar medium containing chloramphenicol were collected as transformants. The genome of the above-obtained transformant was extracted, and PCR performed thereon confirmed that the ccpA gene had been deleted and substituted by a chloramphenicol-resistant gene.

TABLE 2-1

| | | SEQ |
|---|---|---|
| Primer | Nucleotide sequence | ID NO: |
| comA-AF | AAGGATGATAATCCGTCCCGTG | 7 |
| comA-A/CmR | GTTATCCGCTCACAATTCGGATGGTCATCAATCA | 8 |
| | CTAG | |
| comA-B/CmF | CGTCGTGACTGGGAAAACTGCGAAATCAGACGGT | 9 |
| | GTAC | |
| comA-BR | CGTCGCCTATCGGCGGGCAC | 10 |
| yop0-AF | ATGTATATAGGAGGTTGGTGGTATG | 11 |
| yop0-A/CmR | GTTATCCGCTCACAATTCGCTCTGACATGTCAAC | 12 |
| | CTCC | |
| yop0-B/CmF | CGTCGTGACTGGGAAAACAGATGAGAAAGGAGGA | 13 |
| | GAAG | |
| yop0-BR | ATAACTGTTACTATATAATGGCC | 14 |
| treR-AF | GCTGGGGATGACGAATCCGA | 15 |
| treR-A/CmR | GTTATCCGCTCACAATTCTCACCTTCATTATGGA | 16 |
| | CCAC | |
| treR-B/CmF | CGTCGTGACTGGGAAAACCACCGTCTCGACAAAT | 17 |
| | TCCG | |
| treR-BR | GTTGCCAAGCGCGATATAGG | 18 |
| yvbA-AF | TATACAGGGATTATCAGTATTGAGC | 19 |
| yvbA-A/CmR | GTTATCCGCTCACAATTCTTTTCTCCTTGTTGGA | 20 |
| | TCTG | |
| yvbA-B/CmF | CGTCGTGACTGGGAAAACGGGGATAACGATTTAT | 21 |
| | GAAG | |
| yvbA-BR | TTTTGTAATAATGATATGAAGCTAGTGTTG | 22 |
| cspB-AF | ATATCCAGCCCTGCCTCTTC | 23 |
| cspB-A/CmR | CTGTGTGAAATTGTTATCCGCTCACAATTC | 24 |
| | GAAATTTCCTCCTAAAGCGATCATAACG | |
| cspB-B/CmF | GTCGTTTTACAACGTCGTTGACTGGGAAAACCCAC | 25 |
| | AAGCTGCTAACGTTAC | |
| cspB-BR | TCCTGTTTGGGCTCCTGTTG | 26 |
| yvaN-AF | TGTTTATGTATGGCGGCCTGCGGGAC | 27 |
| yvaN-A/CmR | GTTATCCGCTCACAATTCAGCTTTCCATATATCT | 28 |
| | CACC | |
| yvaN-B/CmF | CGTCGTGACTGGGAAAACACGGTCTGCTGATGAC | 29 |
| | TGAC | |
| yvaN-BR | GCGTTTACTTAAGATGTCGA | 30 |
| yttP-AF | TTTCTAGCGTTTCGGCAAATTGAGTTAAG | 31 |
| yttP-A/CmR | GTTATCCGCTCACAATTCCTTACTTTCATACGGC | 32 |
| | TCAC | |
| yttP-B/CmF | CGTCGTGACTGGGAAAACGAGACGTGGCGCTCAC | 33 |
| | CAAC | |
| yttP-BR | CGGATTAAAAAAAGAATATCGCGGACAGC | 34 |
| yurK-AF | TGCCGCTGCCCGCCGGAGAG | 35 |

TABLE 2-2

| yurK-A/CmR | GTTATCCGCTCACAATTCAAGGTGTAGAACTTCCGTTG | 36 |
|---|---|---|
| yurK-B/CmF | CGTCGTGACTGGGAAAACACCATCAACAGCCCCTACAC | 37 |
| yurK-BR | TCAAATAAAGGCGGCATTCAGTCC | 38 |
| yozA-AF | ATAATGGTATCCAAATCCACGC | 39 |
| yozA-A/CmR | GTTATCCGCTCACAATTCATTCAGTCATATGTATCACC | 40 |
| yozA-B/CmF | CGTCGTGACTGGGAAAACGATCCATCATACACAGCATG | 41 |
| yozA-BR | CACTTCTCAACGGAGGGGATTTCACATC | 42 |
| licR-AF | TAATGGAGGAGAGAAGGCCG | 43 |
| licR-A/CmR | GTTATCCGCTCACAATTCAGTCGCCCATGAAGCATGAG | 44 |
| licR-B/CmF | CGTCGTGACTGGGAAAACACCAAAAAATGCTG | 45 |
| | AGCTGACAGC | |
| licR-BR | TTGCCAATGATGAGGAAAAAGGAACC | 46 |
| sigL-AF | CTGAACGTCTTGAATAAAAAAGCAGG | 47 |
| sigL-A/CmR | GTTATCCGCTCACAATTCGCTGAAGTTTCATATCCATC | 48 |
| sigL-B/CmF | CGTCGTGACTGGGAAAACATTCCGTCATCGGCAGCGAG | 49 |
| sigL-BR | AGCGGTTTACAAGTTGGAGG | 50 |
| mntR-AF | ATTTCAGAAGGCATACTTCAAG | 51 |
| mntR-A/CmR | GTTATCCGCTCACAATTCCATACTTGGTGTTGTCATCG | 52 |
| mntR-B/CmF | CGTCGTGACTGGGAAAACCATAATCAGTAAAAA | 53 |
| | GGCGGTC | |
| mntR-BR | TTCTGACCGCTCTGGCAACC | 54 |
| glcT-AF | ATAATGCCCGCTTCCCAACC | 55 |
| glcT-A/CmR | GTTATCCGCTCACAATTCCGATCCTCAGCTCCTTTGTC | 56 |
| glcT-B/CmF | CGTCGTGACTGGGAAAACTCATCTGATACCGATTAACC | 57 |
| glcT-BR | CAACTGAATCCGAAGGAATG | 58 |
| yvdE-AF | TCGGGGTCATGCCGAGCGGT | 59 |
| yvdE-A/CmR | GTTATCCGCTCACAATTCCAATGTTGCCATTTTCATCC | 60 |
| yvdE-B/CmF | CGTCGTGACTGGGAAAACTTGTACGAGAATCAACGCTG | 61 |
| yvdE-BR | CACGGCAATGCATTCTTCGG | 62 |
| ykvE-AF | AGATCTGTCGGCCAGGTTTAC | 63 |
| ykvE-A/CmR | GTTATCCGCTCACAATTCTGATTTTTCTGTCATGTCTC | 64 |
| ykvE-B/CmF | CGTCGTGACTGGGAAAACGGTAGAGATGTGCACCGAAA | 65 |
| ykvE-BR | GAGTCAGACGGCATCGATGA | 66 |
| slr-AF | TTCTGATTCATTTTCACTGCTGG | 67 |
| slr-A/CmR | GTTATCCGCTCACAATTCAACGGATAATTCTTCCAATC | 68 |
| slr-B/CmF | CGTCGTGACTGGGAAAACTGTCCATGAAGTCAAATCC | 69 |
| slr-BR | CGCTGAAATATTCTCTCGCA | 70 |
| rocR-AF | CGCCGCTTTCACCGCGGATTC | 71 |
| rocR-A/CmR | GTTATCCGCTCACAATTCCTTTGACCACTGTATGAACC | 72 |

TABLE 2-3

| | | |
|---|---|---|
| rocR-B/CmF | CGTCGTGACTGGGAAAACACTCGTCTAACGAATAATCC | 73 |
| rocR-BR | TGTCATCACGGAATTTGACG | 74 |
| ccpA-AF | CCAAATTATCCTTTGTGAGCGCGGAATCAG | 75 |
| ccpA-A/CmR | GTTATCCGCTCACAATTCCGTAGATCGTAATATTGCTC | 76 |
| ccpA-B/CmF | CGTCGTGACTGGGAAAACAGCTTAGAAAGTCAACCAAG | 77 |
| ccpA-BR | TTTGAGCATCAGCACAAGCC | 78 |
| yaaT-AF | TGTAGCAGAAGCAGTCGAATT | 79 |
| yaaT-A/Cm2R | CTAATGGGTGCTTTAGTTGACAATTACGCAGCTGTC | 80 |
| | ATGT | |
| yaaT-B/Cm2F | CTGCCCCGTTAGTTGAAGAACTGATAAACCGTGAAA | 81 |
| | AAGTG | |
| yaaT-RV | CCTTTGAAAAAGGCTCCCGT | 82 |
| yyaA-AF | GTTTTCCAAGTCTGCCGATAAAAATATGC | 83 |
| yyaA-A/CmR | GTTATCCGCTCACAATTCATGCTTCATGTACCTACACC | 84 |
| yyaA-B/CmF | CGTCGTGACTGGGAAAACCAATTAACGATTCGCATACC | 85 |
| yyaA-BR | AAAAAGAAGAAGTCACAGTACAGAACGTGG | 86 |
| yycH-AF | ATTTTTCGCCATCTTGAATTTTC | 87 |
| yycH-A/Cm2R | CTAATGGGTGCTTTAGTTGGATGATCCTCTCGTTGA | 88 |
| | ACTG | |
| yycH-B/Cm2F | CTGCCCCGTTAGTTGAAGGGATGAGCCTTCAGAAA | 89 |
| | AGTT | |
| yycH-BR | GCCGGACAGAGATCTGTATG | 90 |
| yacP-B/Cm4F | GAAGAAGGTTTTTATGTTGACGCTTTTTTGCCCAATA | 91 |
| | CTGTATAA | |
| yacP-B/Cm4R | CAAAAAAGCGTCAACATAAAAACCTTCTTCAACTAAC | 92 |
| | GGGGCAGG | |
| yacP-BR | AAGACGAGTACTTTTCTCTCTAAATCACTT | 93 |
| yacP-AF | AACTCGATCAAATGGTGACAGGACAGCATC | 94 |
| yacP-A/Cm4F | GGAGAATAAAGACCCTCTTCAACTAAAGCACCCATTA | 95 |
| | GTTCAACA | |
| yacP-A/Cm4R | TGCTTTAGTTGAAGAGGGTCTTTATTCTCCCACAGGG | 96 |
| | TTTCGTTT | |
| hprK-B/Cm4F | TTTTTATATTACAGCGAGTTGGCGTTAAATGAATGAA | 97 |
| | GCGATAGA | |
| hprK-B/Cm4R | ATTTAACGCCAACTCGCTGTAATATAAAAACCTTCTT | 98 |
| | CAACTAAC | |
| hprK-BR | TTGATTGATGATAAATTCAGGCAGGTGCAG | 99 |
| hprK-AF | CAAAGCTTGAGAAATGTTCCCATGCTCTTG | 100 |
| hprK-A/Cm4F | CAGGAGGAACATATCTCTTCAACTAAAGCACCCATT | 101 |
| | AGTTCAACA | |
| hprK-A/Cm4R | TGCTTTAGTTGAAGAGATATGTTCCTCCTGTTCCGG | 102 |
| | GCTGCCCCG | |
| rsiX-AF | ATTCCAGTTACTCGTAATATAGTTG | 103 |
| rsiX-A/CmR | GTTATCCGCTCACAATTCACTTCATCATCCATTA | 104 |
| | GCTC | |
| rsiX-B/CmF | CGTCGTGACTGGGAAAACCTGCTCCAAATCCGAT | 105 |
| | TTCC | |
| rsiX-BR | GTCCTGCATTTTTCGAAGTCTGG | 106 |
| yhdK-AF | TACACATCCTTCAAACAAGTCTGAACAAAC | 107 |

TABLE 2-4

| | | |
|---|---|---|
| yhdK-A/Cm4R | TGCTTTAGTTGAAGATTACCAGTTCCATAATT | 108 |
| | CCACCTCGCCGAC | |
| yhdK-B/Cm4F | TTTTTATATTACAGCGTGTGTATACCATTGTA | 109 |
| | TCTGTAGATACGA | |
| yhdK-BR | GCTATGATCATTGTAACGAAAGGAAAGGGG | 110 |
| yhdK-A/Cm4F | TTATGGAACTGGTAATCTTCAACTAAAGCACCC | 111 |
| | ATTAGTTCAACA | |
| yhdK-B/Cm4R | CAATGGTATACACACGCTGTAATATAAAAACCT | 112 |
| | TCTTCAACTAAC | |
| y1b0-AF | AATCTGAACAAGAAAAAGGAGCTGCTCCTC | 113 |
| y1b0-A/Cm4R | TGCTTTAGTTGAAGAATTCAATCTCCCTCCAT | 114 |
| | GTCAGCTTATTTA | |
| y1b0-B/Cm4F | TTTTTATATTACAGCAGAAACGCCTGAAATG | 115 |
| | AACCGGCCCTATAG | |
| y1b0-BR | TGTTTGACAAAGGTAGAACGTCTGCTTATC | 116 |
| y1b0-A/Cm4F | GGAGGGAGATTGAATTCTTCAACTAAAGCACCC | 117 |
| | ATTAGTTCAACA | |
| y1b0-B/Cm4R | ATTTCAGGCGTTTCTGCTGTAATATAAAAACC | 118 |
| | TTCTTCAACTAAC | |
| CmF | GAATTGTGAGCGGATAAC | 119 |
| CmR | GTTTTCCCAGTCACGACG | 120 |
| Cm2F | CAACTAAAGCACCCATTAG | 121 |
| Cm2R | CTTCAACTAACGGGGCAG | 122 |

### Example 2

In a manner similar to that described in Example 1, sporulation gene-deleted strains into which a chloramphenicol-resistant gene had been introduced by way of substitution in place of the below-described deleted genes were separated through use of a DNA fragment for effecting deletion prepared from an adequate primer set selected from among various primer sets shown in Table 2; i.e., gene-AF, gene-A/CmR, gene-B/CmF, gene-BR, CmF, and CmR. The gene deleted from the genome was comA, yopO, treR, yvbA, yvaN, yttP, yurK, yozA, licR, sigL, mntR, glcT, ykvE, slr, rocR, yyaA, or rsiX.

### Example 3

In a manner similar to that described in Example 2, a DNA fragment for deletion was prepared by use of an adequate primer set selected from among the gene-AF, gene-A/Cm2R, gene-B/Cm2F, gene-BR, Cm2F, and Cm2R, which are shown in Table 2. By use of the thus-prepared DNA fragment, sporulation gene-deleted strains into which a chloramphenicol-resistant gene had been introduced by way of substitution in place of the below-described deleted genes were separated. The gene deleted from the genome was cspB, yvdE, yaaT, yycH, or ylbO.

### Example 4

In a manner similar to that described in Example 2, a DNA fragment for effecting deletion was prepared from an adequate primer set selected from among the gene-AF, gene-A/Cm4R, gene-B/Cm4F, gene-BR, Cm4F, and Cm4R, which are shown in Table 2. By use of the thus-prepared DNA fragment, sporulation gene-deleted strains into which a chloramphenicol-resistant gene had been introduced by way of substitution in place of deleted genes; yacP, hprK, and yhdK, were separated.

### Example 5

To each of the gene-deleted strains obtained in Examples 1 to 4 and to Bacillus subtilis 168 strain serving as a control, a recombinant plasmid pHY-S237 was introduced through the protoplast transformation method. The recombinant plasmid pHY-S237 was prepared by inserting a DNA fragment (3.1 kb) encoding an alkaline cellulase derived from Bacillus sp. KSM-S237 strain (Japanese Patent Application Laid-Open (kokai) No. 2000-210081) into the restriction enzyme BamHI cleavage site of a shuttle vector pHY300 PLK. Each of the thus-obtained cell strains was shake-cultured in an LB medium (5 mL) overnight at 30° C. The culture broth (0.03 mL) was inoculated to a 2×L-maltose medium (2% tryptone, 1% yeast extract, 1% NaCl, 7.5% maltose, 7.5 ppm manganese sulfate 4-5 hydrate, and 15 ppm tetracycline), followed by shake culturing at 30° C. for three days. After completion of culturing, cells were removed through centrifugation, and alkaline cellulase activity of the supernatant obtained from the culture was determined, thereby calculating the amount of the alkaline cellulase secreted from the cells during culturing; i.e., the amount of the extracellularly produced alkaline cellulase. As is clear from Table 3, more effective production, or secretion, of alkaline cellulase has been confirmed in the case where a gene-deleted strain was employed as a host, as compared with the control 168 strain (wild type strain).

TABLE 3

| | | | Size of | Amount of produced |
|---|---|---|---|---|
| | | Gene | deleted | (secreted) alkaline |
| Name of | | size | fragment | cellulase (relative |
| deleted gene | Gene ID | (bp) | (bp) | value) |
| comA | BG10381 | 645 | 588 | 160 |
| yopO | BG13648 | 213 | 169 | 154 |
| treR | BG11011 | 717 | 656 | 139 |
| yvbA | BG14078 | 273 | 210 | 137 |
| cspB | BG10824 | 204 | 171 | 132 |
| yvaN | BG14069 | 408 | 379 | 124 |
| yttP | BG13927 | 624 | 590 | 121 |
| yurK | BG13997 | 729 | 677 | 118 |
| yozA | BG13748 | 324 | 289 | 117 |
| licR | BG11346 | 1926 | 1889 | 116 |
| sigL | BG10748 | 1311 | 1256 | 114 |
| mntR | BG11702 | 429 | 399 | 114 |
| glcT | BG12593 | 858 | 811 | 110 |
| yvdE | BG12414 | 951 | 916 | 109 |
| ykvE | BG13310 | 438 | 356 | 108 |
| slr | BG11858 | 459 | 394 | 105 |
| rocR | BG10723 | 1386 | 1359 | 128 |
| ccpA | BG10376 | 1005 | 957 | 205 |
| yaaT | BG10096 | 828 | 828 | 127 |
| yyaA | BG10057 | 852 | 816 | 113 |
| yycH | BG11462 | 1368 | 1368 | 146 |
| yacP | BG10158 | 513 | 513 | 156 |
| hprK | BG14125 | 933 | 933 | 196 |
| rsiX | BG10537 | 1107 | 1068 | 125 |
| yhdK | BG13017 | 291 | 228 | 114 |
| ylbO | BG13367 | 582 | 582 | 136 |
| None | — | — | — | 100 |
| (Wild type) | | | | |

### Example 6

To each of the gene-deleted strains obtained in Examples 1 to 4 and to Bacillus subtilis 168 strain serving as a control, recombinant plasmid pHSP-K38 was introduced through the protoplast transformation method. The recombinant plasmid pHSP-K38 was prepared by inserting, into the restriction enzyme BagII-XbaI cleavage site of a shuttle vector pHY300 PLK, a 2.1 kb fragment (SEQ ID No: 5) prepared by ligating an upstream 0.6 kb fragment (SEQ ID NO: 3) including portions of a promoter region and a signal sequence region of an alkaline cellulase gene with an upstream side of a DNA fragment (1.5 kb) encoding a mature enzyme region (Asp1-Gln480) of an alkaline amylase gene derived from Bacillus sp. KSM-K38 strain (Japanese Patent Application Laid-Open (kokai) No. 2000-1884882, Eur. J. Biochem., 268, 2974 (2001)). Each of the thus-obtained cell strains was shake-cultured in an LB medium (5 mL) overnight at 30° C. The culture broth (0.03 mL) was inoculated to a 2×L-maltose medium (2% tryptone, 1% yeast extract, 1% NaCl, 7.5% maltose, 7.5 ppm manganese sulfate 4-5 hydrate, and 15 ppm tetracycline), followed by shake culturing at 30° C. for three to six days. After completion of culturing, cells were removed through centrifugation, and alkaline amylase activity of the supernatant obtained from the culture was determined, thereby calculating the amount of the alkaline amylase secreted from the cells during culturing; i.e., the amount of the extracellularly produced alkaline amylase. As is clear from Table 3, more effective production, or secretion, of alkaline amylase has been confirmed in the case where a gene-deleted strain was employed as a host, as compared with the control 168 strain (wild type strain).

TABLE 4

| | | | | |
|---|---|---|---|---|
| | | | Size of | Amount of produced |
| | | Gene | deleted | (secreted) alkaline |
| Name of | | size | fragment | amylase (relative |
| deleted gene | Gene ID | (bp) | (bp) | value) |

Cultured for 3 days

| | | | | |
|---|---|---|---|---|
| slr | BG11858 | 459 | 394 | 178 |
| treR | BG11011 | 717 | 656 | 124 |
| yopO | BG13648 | 213 | 169 | 364 |
| yvaN | BG14069 | 408 | 379 | 148 |
| yvbA | BG14078 | 273 | 210 | 171 |
| None | — | — | — | 100 |
| (Wild type) | | | | |

Culture for 5 days (Wild type)

| | | | | |
|---|---|---|---|---|
| cspB | BG10824 | 204 | 171 | 195 |
| rocR | BG10723 | 1386 | 1359 | 215 |
| sigL | BG10748 | 1311 | 1256 | 204 |
| glcT | BG12593 | 858 | 811 | 132 |
| yvdE | BG12414 | 951 | 916 | 127 |
| yacP | BG10158 | 513 | 513 | 110 |
| None | — | — | — | 100 |
| (Wild type) | | | | |

Cultured for 6 days

| | | | | |
|---|---|---|---|---|
| yycH | BG11462 | 1368 | 1368 | 120 |
| licR | BG11346 | 1926 | 1889 | 122 |
| None | — | — | — | 100 |
| (Wild type) | | | | |

1-7. (canceled)

8. A recombinant microorganism comprising:
a heterologous polynucleotide that encodes a heterologous protein or polypeptide, and from which
one or more of the following Bacillus subtilis genes have been deleted or knocked-out: yopO, yvbA, yvdE, yacP, a gene having at least 90% homology with the nucleotide sequence of yopO, a gene having at least 90% homology with the nucleotide sequence of yvbA, a gene having at least 90% homology with the nucleotide sequence of yvdE, and a gene having at least 90% homology with the nucleotide sequence of yacP.

9. The recombinant microorganism of claim 8, wherein the microorganism is Bacillus subtilis.

10. The recombinant microorganism of claim 8, wherein one or more regions selected from among a transcription initiation regulatory region, a translation initiation regulatory region, and a secretion signal region is ligated to an upstream region of a gene encoding a heterologous protein or polypeptide.

11. The recombinant microorganism of claim 10, wherein the one or more regions are three regions constituted by a transcription initiation regulatory region, a translation initiation regulatory region, and a secretion signal region.

12. The recombinant microorganism of claim 10, wherein the secretion signal region is derived from a cellulase gene of a bacterium belonging to the genus Bacillus and the transcription initiation regulatory region and the translation initiation regulatory region are each derived from a 0.6 to 1 kb region upstream of the cellulase gene.

13. The recombinant microorganism of claim 11, wherein the three regions constituted by the transcription initiation regulatory region, the translation initiation regulatory region, and the secretion signal region are a nucleotide sequence of base numbers 1 to 659 of a cellulase gene of SEQ ID NO: 1; a nucleotide sequence of base numbers 1 to 696 of a cellulase gene of SEQ ID NO: 3; a DNA fragment having a nucleotide sequence having at least 70% homology with either of these nucleotide sequences; or a DNA fragment having a nucleotide sequence lacking a portion of any one of these nucleotide sequences.

14. The recombinant microorganism of claim 8, wherein Bacillus subtilis yopO gene or a gene having at least 90% homology with the nucleotide sequence of yopO has been deleted or its expression knocked out.

15. The recombinant microorganism of claim 8, wherein Bacillus subtilis yvbA gene or a gene having at least 90% homology with the nucleotide sequence of yvbA has been deleted or its expression knocked out.

16. The recombinant microorganism of claim 8, wherein Bacillus subtilis yvdE gene or a gene having at least 90% homology with the nucleotide sequence of yvdE has been deleted or its expression knocked out.

17. The recombinant microorganism of claim 8, wherein Bacillus subtilis yacP gene or a gene having at least 90% homology with the nucleotide sequence of yacP has been deleted or its expression knocked out.

18. A method for producing a protein or polypeptide comprising:
growing or culturing the recombinant microorganism of claim 8 for a time and under conditions suitable for expression of said heterologous protein or polypeptide, and recovering said heterologous protein or polypeptide.

19. A recombinant microorganism that is Bacillus comprising a heterologous polynucleotide that encodes a heterologous protein or polypeptide,
wherein said microorganism has one or more of the following Bacillus subtilis genes deleted or knocked-out: yopO, yvbA, yvdE, yacP, a gene having at least 90% homology with the nucleotide sequence of yopO, a gene having at least 90% homology with the nucleotide sequence of yvbA, a gene having at least 90% homology with the nucleotide sequence of yvdE, and a gene having at least 90% homology with the nucleotide sequence of yacP.

20. The recombinant microorganism of claim 19, wherein Bacillus subtilis yopO gene or a gene having at least 90% homology with the nucleotide sequence of yopO has been deleted or its expression knocked out.

21. The recombinant microorganism of claim 19, wherein Bacillus subtilis yvbA gene or a gene having at least 90% homology with the nucleotide sequence of yvbA has been deleted or its expression knocked out.

22. The recombinant microorganism of claim 19, wherein Bacillus subtilis yvdE gene or a gene having at least 90% homology with the nucleotide sequence of yvdE has been deleted or its expression knocked out.

23. The recombinant microorganism of claim 19, wherein Bacillus subtilis yacP gene or a gene having at least 90% homology with the nucleotide sequence of yacP has been deleted or its expression knocked out.

24. A method for producing a protein or polypeptide comprising:
growing or culturing the recombinant microorganism of claim 19 for a time and under conditions suitable for expression of said heterologous protein or polypeptide, and recovering said heterologous protein or polypeptide.


SEQUENCE LISTING (122 sequences)

SEQ ID NO: 1 - DNA, 3150 bp, Bacillus sp. KSM-S237
  CDS (573)..(3044)
  sig_peptide (573)..(659)
gatttgccgatgcaacaggcttatatttagaggaaatttctttttaaattgaatacggaataaaatcaggtaaacaggtcctgattttatttttttgagttttttagagaactgaagattgaaataaaagtagaagacaaaggacataagaaaattgcattagttttaattatagaaaacgcctttttataattatttatacctagaacgaaaatactgtttcgaaagcggtttactataaaaccttatattccggctcttttttaaaacagggggtaaaaattcactctagtattctaatttcaacatgctataataaatttgtaagacgcaatatgcatctctttttttacgatatatgtaagcggttaaccttgtgctatatgccgatttaggaaggggggtagattgagtcaagtagtaataatatagataacttataagttgttgagaagcaggagagcatctgggttactcacaagtttttttaaaactttaacgaaagcactttcggtaatgcttatgaatttagctatttgattcaattactttaaaaatatttaggaggtaatatgatgttaagaaagaaaacaaagcagttgatttcttccattcttattttagttttacttctatctttatttccggcagctcttgcagcagaaggaaacactcgtgaagacaattttaaacatttattaggtaatgacaatgttaaacgcccttctgaggctggcgcattacaattacaagaagtcgatggacaaatgacattagtagatcaacatggagaaaaaattcaattacgtggaatgagtacacacggattacagtggtttcctgagatcttgaatgataacgcatacaaagctctttctaacgattgggattccaatatgattcgtcttgctatgtatgtaggtgaaaatgggtacgctacaaaccctgagttaatcaaacaaagagtgattgatggaattgagttagcgattgaaaatgacatgtatgttattgttgactggcatgttcatgcgccaggtgatcctagagatcctgtttatgcaggtgctaaagatttctttagagaaattgcagctttataccctaataatccacacattatttatgagttagcgaatgagccgagtagtaataataatggtggagcagggattccgaataacgaagaaggttggaaagcggtaaaagaatatgctgatccaattgtagaaatgttacgtaaaagcggtaatgcagatgacaacattatcattgttggtagtccaaactggagtcagcgtccggacttagcagctgataatccaattgatgatcaccatacaatgtatactgttcacttctacactggttcacatgctgcttcaactgaaagctatccgtctgaaactcctaactctgaaagaggaaacgtaatgagtaacactcgttatgcgttagaaaacggagtagcggtatttgcaacagagtggggaacgagtcaagctagtggagacggtggtccttactttgatgaagcagatgtatggattgaatttttaaatgaaaacaacattagctgggctaactggtctttaacgaataaaaatgaagtatctggtgcatttacaccattcgagttaggtaagtctaacgcaaccaatcttgacccaggtccagatcatgtgtgggcaccagaagaattaagtctttctggagaatatgtacgtgctcgtattaaaggtgtgaactatgagccaatcgaccgtacaaaatacacgaaagtactttgggactttaatgatggaacgaagcaaggatttggagtgaattcggattctccaaataaagaacttattgcagttgataatgaaaacaacactttgaaagtttcgggattagatgtaagtaacgatgtttcagatggcaacttctgggctaatgctcgtctttctgccaacggttggggaaaaagtgttgatattttaggtgctgagaagcttacaatggatgttattgttgatgaaccaacgacggtagctattgcggcgattccacaaagtagtaaaagtggatgggcaaatccagagcgtgctgttcgagtgaacgcggaagattttgtccagcaaacggacggtaagtataaagctggattaacaattacaggagaagatgctcctaacctaaaaaatatcgcttttcatgaagaagataacaatatgaacaacatcattctgttcgtgggaactgatgcagctgacgttatttacttagataacattaaagtaattggaacagaagttgaaattccagttgttcatgatccaaaaggagaagctgttcttccttctgtttttgaagacggtacacgtcaaggttgggactgggctggagagtctggtgtgaaaacagctttaacaattgaagaagcaaacggttctaacgcgttatcatgggaatttggatatccagaagtaaaacctagtgataactgggcaacagctccacgtttagatttctggaaatctgacttggttcgcggtgagaatgattatgtagcttttgatttctatctagatccagttcgtgcaacagaaggcgcaatgaatatcaatttagtattccagccacctactaacgggtattgggtacaagcaccaaaaacgtatacgattaactttgatgaattagaggaagcgaatcaagtaaatggtttatatcactatgaagtgaaaattaacgtaagagatattacaaacattcaagatgacacgttactacgtaacatgatgatcatttttgcagatgtagaaagtgactttgcagggagagtctttgtagataatgttcgttttgagggggctgctactactgagccggttgaaccagagccagttgatcctggcgaagagacgccacctgtcgatgagaaggaagcgaaaaaagaacaaaaagaagcagagaaagaagagaaagaagcagtaaaagaagaaaagaaagaagctaaagaagaaaagaaagcagtcaaaaatgaggctaagaaaaaataatctattaaactagttatagggttatctaaaggtctgatgtagatcttttagataacctttttcttgcataactggacacagagttgttattaaagaaagtaag

SEQ ID NO: 2 - protein, 824 aa, Bacillus sp. KSM-S237
MMLRKKTKQLISSILILVLLLSLFPAALAAEGNTREDNFKHLLGNDNVKRPSEAGALQLQEVDGQMTLVDQHGEKIQLRGMSTHGLQWFPEILNDNAYKALSNDWDSNMIRLAMYVGENGYATNPELIKQRVIDGIELAIENDMYVIVDWHVHAPGDPRDPVYAGAKDFFREIAALYPNNPHIIYELANEPSSNNNGGAGIPNNEEGWKAVKEYADPIVEMLRKSGNADDNIIIVGSPNWSQRPDLAADNPIDDHHTMYTVHFYTGSHAASTESYPSETPNSERGNVMSNTRYALENGVAVFATEWGTSQASGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSANGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEAVKEEKKEAKEEKKAVKNEAKKK

SEQ ID NO: 3 - DNA, 3332 bp, Bacillus sp. KSM-64
  CDS (610)..(3075)
  sig_peptide (610)..(696)
agtacttaccattttagagtcaaaagatagaagccaagcaggatttgccgatgcaaccggcttatatttagagggaatttctttttaaattgaatacggaataaaatcaggtaaacaggtcctgattttatttttttgaatttttttgagaactaaagattgaaatagaagtagaagacaacggacataagaaaattgtattagttttaattatagaaaacgcttttctataattatttatacctagaacgaaaatactgtttcgaaagcggtttactataaaaccttatattccggctctttttttaaacagggggtgaaaattcactctagtattctaatttcaacatgctataataaatttgtaagacgcaatatacatcttttttttatgatatttgtaagcggttaaccttgtgctatatgccgatttaggaagggggtagattgagtcaagtagtcataatttagataacttataagttgttgagaagcaggagagaatctgggttactcacaagttttttaaaacattatcgaaagcactttcggttatgcttatgaatttagctatttgattcaattactttaataattttaggaggtaatatgatgttaagaaagaaaacaaagcagttgatttcttccattcttattttagttttacttctatctttatttccgacagctcttgcagcagaaggaaacactcgtgaagacaattttaaacatttattaggtaatgacaatgttaaacgcccttctgaggctggcgcattacaattacaagaagtcgatggacaaatgacattagtagatcaacatggagaaaaaattcaattacgtggaatgagtacacacggattacaatggtttcctgagatcttgaatgataacgcatacaaagctcttgctaacgattgggaatcaaatatgattcgtctagctatgtatgtcggtgaaaatggctatgcttcaaatccagagttaattaaaagcagagtcattaaaggaatagatcttgctattgaaaatgacatgtatgtcatcgttgattggcatgtacatgcacctggtgatcctagagatcccgtttacgctggagcagaagatttctttagagatattgcagcattatatcctaacaatccacacattatttatgagttagcgaatgagccaagtagtaacaataatggtggagctgggattccaaataatgaagaaggttggaatgcggtaaaagaatacgctgatccaattgtagaaatgttacgtgatagcgggaacgcagatgacaatattatcattgtgggtagtccaaactggagtcagcgtcctgacttagcagctgataatccaattgatgatcaccatacaatgtatactgttcacttctacactggttcacatgctgcttcaactgaaagctatccgcctgaaactcctaactctgaaagaggaaacgtaatgagtaacactcgttatgcgttagaaaacggagtagcagtatttgcaacagagtggggaactagccaagcaaatggagatggtggtccttactttgatgaagcagatgtatggattgagtttttaaatgaaaacaacattagctgggctaactggtctttaacgaataaaaatgaagtatctggtgcatttacaccattcgagttaggtaagtctaacgcaacaagtcttgacccagggccagaccaagtatgggtaccagaagagttaagtctttctggagaatatgtacgtgctcgtattaaaggtgtgaactatgagccaatcgaccgtacaaaatacacgaaagtactttgggactttaatgatggaacgaagcaaggatttggagtgaatggagattctccagttgaagatgtagttattgagaatgaagcgggcgctttaaaactttcaggattagatgcaagtaatgatgtttctgaaggtaattactgggctaatgctcgtctttctgccgacggttggggaaaaagtgttgatattttaggtgctgaaaaacttactatggatgtgattgttgatgagccgaccacggtatcaattgctgcaattccacaagggccatcagccaattgggttaatccaaatcgtgcaattaaggttgagccaactaatttcgtaccgttaggagataagtttaaagcggaattaactataacttcagctgactctccatcgttagaagctattgcgatgcatgctgaaaataacaacatcaacaacatcattctttttgtaggaactgaaggtgctgatgttatctatttagataacattaaagtaattggaacagaagttgaaattccagttgttcatgatccaaaaggagaagctgttcttccttctgtttttgaagacggtacacgtcaaggttgggactgggctggagagtctggtgtgaaaacagctttaacaattgaagaagcaaacggttctaacgcgttatcatgggaatttggatacccagaagtaaaacctagtgataactgggcaacagctccacgtttagatttctggaaatctgacttggttcgcggtgaaaatgattatgtaacttttgatttctatctagatccagttcgtgcaacagaaggcgcaatgaatatcaatttagtattccagccacctactaacgggtattgggtacaagcaccaaaaacgtatacgattaactttgatgaattagaggaagcgaatcaagtaaatggtttatatcactatgaagtgaaaattaacgtaagagatattacaaacattcaagatgacacgttactacgtaacatgatgatcatttttgcagatgtagaaagtgactttgcagggagagtctttgtagataatgttcgttttgagggggctgctactactgagccggttgaaccagagccagttgatcctggcgaagagacgccgcctgtcgatgagaaggaagcgaaaaaagaacaaaaagaagcagagaaagaagagaaagaagcagtaaaagaagaaaagaaagaagctaaagaagaaaagaaagcaatcaaaaatgaggctacgaaaaaataatctaataaactagttatagggttatctaaaggtctgatgcagatcttttagataacctttttttgcataactggacatagaatggttattaaagaaagcaaggtgtttatacgatattaaaaaggtagcgattttaaattgaaacctttaataatgtcttgtgatagaatgatgaagtaatttaagagggggaaacgaagtgaaaacggaaatttctagtagaagaaaaacagaccaagaaatactgcaagctt

SEQ ID NO: 4 - protein, 822 aa, Bacillus sp. KSM-64
MMLRKKTKQLISSILILVLLLSLFPTALAAEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPIDDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATSLDPGPDQVWVPEELSLSGEYVRARIKGVNYEPIDRTKYTKVLWDFNDGTKQGFGVNGDSPVEDVVIENEAGALKLSGLDASNDVSEGNYWANARLSADGWGKSVDILGAEKLTMDVIVDEPTTVSIAAIPQGPSANWVNPNRAIKVEPTNFVPLGDKFKAELTITSADSPSLEAIAMHAENNNINNIILFVGTEGADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVTFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEAVKEEKKEAKEEKKAIKNEATKK

SEQ ID NO: 5 - DNA, 2343 bp, Bacillus sp. pHSP-K38
  CDS (580)..(2067)
  sig_peptide (580)..(627)
agatctagcaggatttgccgatgcaaccggcttatatttagagggaatttctttttaaattgaatacggaataaaatcaggtaaacaggtcctgattttatttttttgaatttttttgagaactaaagattgaaatagaagtagaagacaacggacataagaaaattgtattagttttaattatagaaaacgcttttctataattatttatacctagaacgaaaatactgtttcgaaagcggtttactataaaaccttatattccggctctttttttaaacagggggtgaaaattcactctagtattctaatttcaacatgctataataaatttgtaagacgcaatatacatcttttttttatgatatttgtaagcggttaaccttgtgctatatgccgatttaggaagggggtagattgagtcaagtagtcataatttagataacttataagttgttgagaagcaggagagaatctgggttactcacaagttttttaaaacattatcgaaagcactttcggttatgcttatgaatttagctatttgattcaattactttaataattttaggaggtaatatgatgttaagaaagaaaacaaagcagttgggtcgaccagcacaagccgatggattgaacggtacgatgatgcagtattatgagtggcatttggaaaacgacgggcagcattggaatcggttgcacgatgatgccgcagctttgagtgatgctggtattacagctatttggattccgccagcctacaaaggtaatagtcaggcggatgttgggtacggtgcatacgatctttatgatttaggagagttcaatcaaaagggtactgttcgaacgaaatacggaactaaggcacagcttgaacgagctattgggtcccttaaatctaatgatatcaatgtatacggagatgtcgtgatgaatcataaaatgggagctgattttacggaggcagtgcaagctgttcaagtaaatccaacgaatcgttggcaggatatttcaggtgcctacacgattgatgcgtggacgggtttcgacttttcagggcgtaacaacgcctattcagattttaagtggagatggttccattttaatggtgttgactgggatcagcgctatcaagaaaatcatattttccgctttgcaaatacgaactggaactggcgagtggatgaagagaacggtaattatgattacctgttaggatcgaatatcgactttagtcatccagaagtacaagatgagttgaaggattggggtagctggtttaccgatgagttagatttggatggttatcgtttagatgctattaaacatattccattctggtatacatctgattgggttcggcatcagcgcaacgaagcagatcaagatttatttgtcgtaggggaatattggaaggatgacgtaggtgctctcgaattttatttagatgaaatgaattgggagatgtctctattcgatgttccacttaattataatttttaccgggcttcacaacaaggtggaagctatgatatgcgtaatattttacgaggatctttagtagaagcgcatccgatgcatgcagttacgtttgttgataatcatgatactcagccaggggagtcattagagtcatgggttgctgattggtttaagccacttgcttatgcgacaattttgacgcgtgaaggtggttatccaaatgtattttacggtgattactatgggattcctaacgataacatttcagctaaaaaagatatgattgatgagctgcttgatgcacgtcaaaattacgcatatggcacgcagcatgactattttgatcattgggatgttgtaggatggactagggaaggatcttcctccagacctaattcaggccttgcgactattatgtcgaatggacctggtggttccaagtggatgtatgtaggacgtcagaatgcaggacaaacatggacagatttaactggtaataacggagcgtccgttacaattaatggcgatggatggggcgaattctttacgaatggaggatctgtatccgtgtacgtgaaccaataacaaaaagccttgagaagggattcctccctaactcaaggctttctttatgtcgcttagctttacgcttctacgactttgaagcttggggatccgtcgagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatgtctaga

SEQ ID NO: 6 - protein, 496 aa, Bacillus sp. pHSP-K38
MMLRKKTKQLGRPAQADGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 7 - DNA, 22 bp, Artificial
  Synthetic DNA
aaggatgataatccgtcccgtg

SEQ ID NO: 8 - DNA, 38 bp, Artificial
  Synthetic DNA
gttatccgctcacaattcggatggtcatcaatcactag

SEQ ID NO: 9 - DNA, 38 bp, Artificial
  Synthetic DNA
cgtcgtgactgggaaaactgcgaaatcagacggtgtac

SEQ ID NO: 10 - DNA, 20 bp, Artificial
  Synthetic DNA
cgtcgcctatcggcgggcac

SEQ ID NO: 11 - DNA, 25 bp, Artificial
  Synthetic DNA
atgtatataggaggttggtggtatg

SEQ ID NO: 12 - DNA, 38 bp, Artificial
  Synthetic DNA
gttatccgctcacaattcgctctgacatgtcaacctcc

SEQ ID NO: 13 - DNA, 38 bp, Artificial
  Synthetic DNA
cgtcgtgactgggaaaacagatgagaaaggaggagaag

SEQ ID NO: 14 - DNA, 23 bp, Artificial
  Synthetic DNA
ataactgttactatataatggcc

SEQ ID NO: 15 - DNA, 20 bp, Artificial
  Synthetic DNA
gctggggatgacgaatccga

SEQ ID NO: 16 - DNA, 38 bp, Artificial
  Synthetic DNA
gttatccgctcacaattctcaccttcattatggaccac

SEQ ID NO: 17 - DNA, 38 bp, Artificial
  Synthetic DNA
cgtcgtgactgggaaaaccaccgtctcgacaaattccg

SEQ ID NO: 18 - DNA, 20 bp, Artificial
  Synthetic DNA
gttgccaagcgcgatatagg

SEQ ID NO: 19 - DNA, 25 bp, Artificial
  Synthetic DNA
tatacagggattatcagtattgagc

SEQ ID NO: 20 - DNA, 38 bp, Artificial
  Synthetic DNA
gttatccgctcacaattcttttctccttgttggatctg

SEQ ID NO: 21 - DNA, 38 bp, Artificial
  Synthetic DNA
cgtcgtgactgggaaaacggggataacgatttatgaag

SEQ ID NO: 22 - DNA, 30 bp, Artificial
  Synthetic DNA
ttttgtaataatgatatgaagctagtgttg

SEQ ID NO: 23 - DNA, 20 bp, Artificial
  Synthetic DNA
atatccagccctgcctcttc

SEQ ID NO: 24 - DNA, 58 bp, Artificial
  Synthetic DNA
ctgtgtgaaattgttatccgctcacaattcgaaatttcctcctaaagcgatcataacg

SEQ ID NO: 25 - DNA, 51 bp, Artificial
  Synthetic DNA
gtcgttttacaacgtcgttgactgggaaaacccacaagctgctaacgttac

SEQ ID NO: 26 - DNA, 20 bp, Artificial
  Synthetic DNA
tcctgtttgggctcctgttg

SEQ ID NO: 27 - DNA, 26 bp, Artificial
  Synthetic DNA
tgtttatgtatggcggcctgcgggac

SEQ ID NO: 28 - DNA, 38 bp, Artificial
  Synthetic DNA
gttatccgctcacaattcagctttccatatatctcacc

SEQ ID NO: 29 - DNA, 38 bp, Artificial
  Synthetic DNA
cgtcgtgactgggaaaacacggtctgctgatgactgac

SEQ ID NO: 30 - DNA, 20 bp, Artificial
  Synthetic DNA
gcgtttacttaagatgtcga

SEQ ID NO: 31 - DNA, 29 bp, Artificial
  Synthetic DNA
tttctagcgtttcggcaaattgagttaag

SEQ ID NO: 32 - DNA, 38 bp, Artificial
  Synthetic DNA
gttatccgctcacaattccttactttcatacggctcac

SEQ ID NO: 33 - DNA, 38 bp, Artificial
  Synthetic DNA
cgtcgtgactgggaaaacgagacgtggcgctcaccaac

SEQ ID NO: 34 - DNA, 29 bp, Artificial
  Synthetic DNA
cggattaaaaaaagaatatcgcggacagc

SEQ ID NO: 35 - DNA, 20 bp, Artificial
  Synthetic DNA
tgccgctgcccgccggagag

SEQ ID NO: 36 - DNA, 38 bp, Artificial
  Synthetic DNA
gttatccgctcacaattcaaggtgtagaacttccgttg

SEQ ID NO: 37 - DNA, 38 bp, Artificial
  Synthetic DNA
cgtcgtgactgggaaaacaccatcaacagcccctacac

SEQ ID NO: 38 - DNA, 24 bp, Artificial
  Synthetic DNA
tcaaataaaggcggcattcagtcc

SEQ ID NO: 39 - DNA, 22 bp, Artificial
  Synthetic DNA
ataatggtatccaaatccacgc

SEQ ID NO: 40 - DNA, 38 bp, Artificial
  Synthetic DNA
gttatccgctcacaattcattcagtcatatgtatcacc

SEQ ID NO: 41 - DNA, 38 bp, Artificial
  Synthetic DNA
cgtcgtgactgggaaaacgatccatcatacacagcatg

SEQ ID NO: 42 - DNA, 28 bp, Artificial
  Synthetic DNA
cacttctcaacggaggggatttcacatc

SEQ ID NO: 43 - DNA, 20 bp, Artificial
  Synthetic DNA
taatggaggagagaaggccg

SEQ ID NO: 44 - DNA, 38 bp, Artificial
  Synthetic DNA
gttatccgctcacaattcagtcgcccatgaagcatgag

SEQ ID NO: 45 - DNA, 42 bp, Artificial
  Synthetic DNA
cgtcgtgactgggaaaacaccaaaaaatgctgagctgacagc

SEQ ID NO: 46 - DNA, 26 bp, Artificial
  Synthetic DNA
ttgccaatgatgaggaaaaaggaacc

SEQ ID NO: 47 - DNA, 26 bp, Artificial
  Synthetic DNA
ctgaacgtcttgaataaaaaagcagg

SEQ ID NO: 48 - DNA, 38 bp, Artificial
  Synthetic DNA
gttatccgctcacaattcgctgaagtttcatatccatc

SEQ ID NO: 49 - DNA, 38 bp, Artificial
  Synthetic DNA
cgtcgtgactgggaaaacattccgtcatcggcagcgag

SEQ ID NO: 50 - DNA, 20 bp, Artificial
  Synthetic DNA
agcggtttacaagttggagg

SEQ ID NO: 51 - DNA, 22 bp, Artificial
  Synthetic DNA
atttcagaaggcatacttcaag

SEQ ID NO: 52 - DNA, 38 bp, Artificial
  Synthetic DNA
gttatccgctcacaattccatacttggtgttgtcatcg

SEQ ID NO: 53 - DNA, 40 bp, Artificial
  Synthetic DNA
cgtcgtgactgggaaaaccataatcagtaaaaaggcggtc

SEQ ID NO: 54 - DNA, 20 bp, Artificial
  Synthetic DNA
ttctgaccgctctggcaacc

SEQ ID NO: 55 - DNA, 20 bp, Artificial
  Synthetic DNA
ataatgcccgcttcccaacc

SEQ ID NO: 56 - DNA, 38 bp, Artificial
  Synthetic DNA
gttatccgctcacaattccgatcctcagctcctttgtc

SEQ ID NO: 57 - DNA, 38 bp, Artificial
  Synthetic DNA
cgtcgtgactgggaaaactcatctgataccgattaacc

SEQ ID NO: 58 - DNA, 20 bp, Artificial
  Synthetic DNA
caactgaatccgaaggaatg

SEQ ID NO: 59 - DNA, 20 bp, Artificial
  Synthetic DNA
tcggggtcatgccgagcggt

SEQ ID NO: 60 - DNA, 38 bp, Artificial
  Synthetic DNA
gttatccgctcacaattccaatgttgccattttcatcc

SEQ ID NO: 61 - DNA, 38 bp, Artificial
  Synthetic DNA
cgtcgtgactgggaaaacttgtacgagaatcaacgctg

SEQ ID NO: 62 - DNA, 20 bp, Artificial
  Synthetic DNA
cacggcaatgcattcttcgg

SEQ ID NO: 63 - DNA, 21 bp, Artificial
  Synthetic DNA
agatctgtcggccaggtttac

SEQ ID NO: 64 - DNA, 38 bp, Artificial
  Synthetic DNA
gttatccgctcacaattctgatttttctgtcatgtctc

SEQ ID NO: 65 - DNA, 38 bp, Artificial
  Synthetic DNA
cgtcgtgactgggaaaacggtagagatgtgcaccgaaa

SEQ ID NO: 66 - DNA, 20 bp, Artificial
  Synthetic DNA
gagtcagacggcatcgatga

SEQ ID NO: 67 - DNA, 23 bp, Artificial
  Synthetic DNA
ttctgattcattttcactgctgg

SEQ ID NO: 68 - DNA, 38 bp, Artificial
  Synthetic DNA
gttatccgctcacaattcaacggataattcttccaatc

SEQ ID NO: 69 - DNA, 37 bp, Artificial
  Synthetic DNA
cgtcgtgactgggaaaactgtccatgaagtcaaatcc

SEQ ID NO: 70 - DNA, 20 bp, Artificial
  Synthetic DNA
cgctgaaatattctctcgca

SEQ ID NO: 71 - DNA, 21 bp, Artificial
  Synthetic DNA
cgccgctttcaccgcggattc

SEQ ID NO: 72 - DNA, 38 bp, Artificial
  Synthetic DNA
gttatccgctcacaattcctttgaccactgtatgaacc

SEQ ID NO: 73 - DNA, 38 bp, Artificial
  Synthetic DNA
cgtcgtgactgggaaaacactcgtctaacgaataatcc

SEQ ID NO: 74 - DNA, 20 bp, Artificial
  Synthetic DNA
tgtcatcacggaatttgacg

SEQ ID NO: 75 - DNA, 30 bp, Artificial
  Synthetic DNA
ccaaattatcctttgtgagcgcggaatcag

SEQ ID NO: 76 - DNA, 38 bp, Artificial
  Synthetic DNA
gttatccgctcacaattccgtagatcgtaatattgctc

SEQ ID NO: 77 - DNA, 38 bp, Artificial
  Synthetic DNA
cgtcgtgactgggaaaacagcttagaaagtcaaccaag

SEQ ID NO: 78 - DNA, 20 bp, Artificial
  Synthetic DNA
tttgagcatcagcacaagcc

SEQ ID NO: 79 - DNA, 21 bp, Artificial
  Synthetic DNA
tgtagcagaagcagtcgaatt

SEQ ID NO: 80 - DNA, 40 bp, Artificial
  Synthetic DNA
ctaatgggtgctttagttgacaattacgcagctgtcatgt

SEQ ID NO: 81 - DNA, 41 bp, Artificial
  Synthetic DNA
ctgccccgttagttgaagaactgataaaccgtgaaaaagtg

SEQ ID NO: 82 - DNA, 20 bp, Artificial
  Synthetic DNA
cctttgaaaaaggctcccgt

SEQ ID NO: 83 - DNA, 29 bp, Artificial
  Synthetic DNA
gttttccaagtctgccgataaaaatatgc

SEQ ID NO: 84 - DNA, 38 bp, Artificial
  Synthetic DNA
gttatccgctcacaattcatgcttcatgtacctacacc

SEQ ID NO: 85 - DNA, 38 bp, Artificial
  Synthetic DNA
cgtcgtgactgggaaaaccaattaacgattcgcatacc

SEQ ID NO: 86 - DNA, 30 bp, Artificial
  Synthetic DNA
aaaaagaagaagtcacagtacagaacgtgg

SEQ ID NO: 87 - DNA, 23 bp, Artificial
  Synthetic DNA
atttttcgccatcttgaattttc

SEQ ID NO: 88 - DNA, 40 bp, Artificial
  Synthetic DNA
ctaatgggtgctttagttggatgatcctctcgttgaactg

SEQ ID NO: 89 - DNA, 39 bp, Artificial
  Synthetic DNA
ctgccccgttagttgaagggatgagccttcagaaaagtt

SEQ ID NO: 90 - DNA, 20 bp, Artificial
  Synthetic DNA
gccggacagagatctgtatg

SEQ ID NO: 91 - DNA, 45 bp, Artificial
  Synthetic DNA
gaagaaggtttttatgttgacgcttttttgcccaatactgtataa

SEQ ID NO: 92 - DNA, 45 bp, Artificial
  Synthetic DNA
caaaaaagcgtcaacataaaaaccttcttcaactaacggggcagg

SEQ ID NO: 93 - DNA, 30 bp, Artificial
  Synthetic DNA
aagacgagtacttttctctctaaatcactt

SEQ ID NO: 94 - DNA, 30 bp, Artificial
  Synthetic DNA
aactcgatcaaatggtgacaggacagcatc

SEQ ID NO: 95 - DNA, 45 bp, Artificial
  Synthetic DNA
ggagaataaagaccctcttcaactaaagcacccattagttcaaca

SEQ ID NO: 96 - DNA, 45 bp, Artificial
  Synthetic DNA
tgctttagttgaagagggtctttattctcccacagggtttcgttt

SEQ ID NO: 97 - DNA, 45 bp, Artificial
  Synthetic DNA
tttttatattacagcgagttggcgttaaatgaatgaagcgataga

SEQ ID NO: 98 - DNA, 45 bp, Artificial
  Synthetic DNA
atttaacgccaactcgctgtaatataaaaaccttcttcaactaac

SEQ ID NO: 99 - DNA, 30 bp, Artificial
  Synthetic DNA
ttgattgatgataaattcaggcaggtgcag

SEQ ID NO: 100 - DNA, 30 bp, Artificial
  Synthetic DNA
caaagcttgagaaatgttcccatgctcttg

SEQ ID NO: 101 - DNA, 45 bp, Artificial
  Synthetic DNA
caggaggaacatatctcttcaactaaagcacccattagttcaaca

SEQ ID NO: 102 - DNA, 45 bp, Artificial
  Synthetic DNA
tgctttagttgaagagatatgttcctcctgttccgggctgccccg

SEQ ID NO: 103 - DNA, 25 bp, Artificial
  Synthetic DNA
attccagttactcgtaatatagttg

SEQ ID NO: 104 - DNA, 38 bp, Artificial
  Synthetic DNA
gttatccgctcacaattcacttcatcatccattagctc

SEQ ID NO: 105 - DNA, 38 bp, Artificial
  Synthetic DNA
cgtcgtgactgggaaaacctgctccaaatccgatttcc

SEQ ID NO: 106 - DNA, 23 bp, Artificial
  Synthetic DNA
gtcctgcatttttcgaagtctgg

SEQ ID NO: 107 - DNA, 30 bp, Artificial
  Synthetic DNA
tacacatccttcaaacaagtctgaacaaac

SEQ ID NO: 108 - DNA, 45 bp, Artificial
  Synthetic DNA
tgctttagttgaagattaccagttccataattccacctcgccgac

SEQ ID NO: 109 - DNA, 45 bp, Artificial
  Synthetic DNA
tttttatattacagcgtgtgtataccattgtatctgtagatacga

SEQ ID NO: 110 - DNA, 30 bp, Artificial
  Synthetic DNA
gctatgatcattgtaacgaaaggaaagggg

SEQ ID NO: 111 - DNA, 45 bp, Artificial
  Synthetic DNA
ttatggaactggtaatcttcaactaaagcacccattagttcaaca

SEQ ID NO: 112 - DNA, 45 bp, Artificial
  Synthetic DNA
caatggtatacacacgctgtaatataaaaaccttcttcaactaac

SEQ ID NO: 113 - DNA, 30 bp, Artificial
  Synthetic DNA
aatctgaacaagaaaaaggagctgctcctc

SEQ ID NO: 114 - DNA, 45 bp, Artificial
  Synthetic DNA
tgctttagttgaagaattcaatctccctccatgtcagcttattta

SEQ ID NO: 115 - DNA, 45 bp, Artificial
  Synthetic DNA
tttttatattacagcagaaacgcctgaaatgaaccggccctatag

SEQ ID NO: 116 - DNA, 30 bp, Artificial
  Synthetic DNA
tgtttgacaaaggtagaacgtctgcttatc

SEQ ID NO: 117 - DNA, 45 bp, Artificial
  Synthetic DNA
ggagggagattgaattcttcaactaaagcacccattagttcaaca

SEQ ID NO: 118 - DNA, 45 bp, Artificial
  Synthetic DNA
atttcaggcgtttctgctgtaatataaaaaccttcttcaactaac

SEQ ID NO: 119 - DNA, 18 bp, Artificial
  Synthetic DNA
gaattgtgagcggataac

SEQ ID NO: 120 - DNA, 18 bp, Artificial
  Synthetic DNA
gttttcccagtcacgacg

SEQ ID NO: 121 - DNA, 19 bp, Artificial
  Synthetic DNA
caactaaagcacccattag

SEQ ID NO: 122 - DNA, 18 bp, Artificial
  Synthetic DNA
cttcaactaacggggcag